(19) United States
(12) Patent Application Publication
(10) Pub. No.: US 2026/0240759 A1
(43) Pub. Date: Aug. 20, 2026

(54) METHODS AND APPARATUSES FOR DELIVERING AN AGENT THROUGH THE VASA VASORUM

(71) Applicant: RenovoRx, Inc., Mountain View, CA (US)

(72) Inventors: Ramtin AGAH, Menlo Park, CA (US); Robert STRASSER, San Jose, CA (US)

(21) Appl. No.: 19/129,277

(22) PCT Filed: Nov. 13, 2023
(86) PCT No.: PCT/US2023/037221

§ 371 Date: May 12, 2025

Related U.S. Application Data
(60) Provisional application No. 63/424,842, filed on Nov. 11, 2022.
(60) Provisional application No. 63/512,903, filed on Jul. 10, 2023.

(51) Int. Cl. A61K 9/00; A61B 90/00; A61K 31/21; A61K 45/06; A61M 25/10

(52) U.S. Cl. CPC A61K 9/0019; A61B 90/37; A61K 31/21; A61K 45/06; A61M 25/1011; A61B 2090/3735

(57) ABSTRACT

Described herein are methods and apparatuses (e.g., devices, systems, etc.) that may be used to deliver one or more agents (e.g., compounds, compositions, etc. including drugs, biologics, cells, etc.) to a target tissue in or adjacent to an adventitia through the vasa vasorum. These methods and apparatuses may be used to deliver relatively large agents to a target tissue in or adjacent to an adventitia specifically through the vasa vasorum.

### CLAIM OF PRIORITY

This patent application claims priority to U.S. Provisional Patent Application No. 63/424,842, titled “METHODS AND APPARATUSES FOR DELIVERING AN AGENT THROUGH THE VASA VASORUM,” filed on Nov. 11, 2022, and U.S. Provisional Patent Application No. 63/512,903, titled “METHODS AND APPARATUSES FOR DELIVERING AN AGENT THROUGH THE VASA VASORUM,” filed on Jul. 10, 2023, each of which is herein incorporated by reference in its entirety.

### BACKGROUND

The vasa vasorum are a microvascular network supporting the outer wall of some blood vessels. Although these dynamic microvessels have been studied for centuries, the importance and impact of their functions in vascular health and disease are not yet fully realized. Recent advances in vasa vasorum imaging for understanding cardiovascular disease severity and pathophysiology open the door for theranostic opportunities. To date, little is known about how to specifically access and employ the vasa vasorum.

In particular, it would be useful to deliver a therapeutic compound (e.g., drug, biologics, cell, etc.) by recruiting the vasa vasorum in order to provide rapid and efficient access to previously inaccessible regions that may be difficult to access due to lack of natural feeder vessels (i.e. arterial branches to the region).

### SUMMARY OF THE DISCLOSURE

Described herein are methods and apparatuses (e.g., devices and systems, including hardware, software and firmware, etc.) that may be used to deliver one or more agents (e.g., compounds, compositions, etc. including drugs, biologics, cells, etc.) to a target tissue in or adjacent to an adventitia specifically through the vasa vasorum. In particular, these methods and apparatuses may be specifically configured to deliver one or more agents from with a vessel (artery or vein) into the tissue through the vasa vasorum without damaging the vasa vasorum, so that relatively large particles can be quickly and effectively delivered to the tissue specifically through the vasa vasorum. Any of these methods and apparatuses may be configured to target the vasa vasorum. In any of these methods and apparatuses, a region of a vessel (e.g., artery) including a sufficient density of vasa vasorum may be occluded, and an agent may be applied into the occluded region in a manner that enhances delivery of the agent through the vasa vasorum and into the adventitia and/or target region. The methods and apparatuses described herein may take advantage of the surprising finding that in some vessel regions there is a direct connection from a lumen of the vessel to the surrounding adventitia, and that, when applied in a manner that optimizes the transport through the vasa vasorum, may allow agents (e.g., drugs) across the vessel wall into surrounding tissue via these channels.

These methods and apparatuses may be used to treat any appropriate tissue, including, but not limited to cancerous tissue. For examples, these apparatuses and methods may relate to the treatment of cancerous tumors or any other target tissue. In some embodiments, the method comprises: identifying a region of a vessel near (e.g., adjacent) to a target tissue that has vasa vasorum; administering a dose (and in particular a therapeutically effective dose) of an agent to an isolated arterial section near the target region that includes vasa vasorum. In any of these methods and apparatuses, the density and/or diameter (e.g., average diameter, etc.) of the vasa vasorum may be determined and the agent may be delivered by occulting a region of artery that is both sufficiently dense in vasa vasorum and sufficiently close to the target region (e.g., is fed by the vasa vasorum within the artery).

In some examples, the method comprises: identifying a region of a vessel (e.g., artery) that is both sufficiently close to a target region (e.g., tumor) and includes a sufficient density of sufficiently sized diameter vasa vasorum so as to deliver a target agent; occluding the identified region of the vessel, and delivering the agent within the occluded region, including under sufficient pressure (e.g., static fluid pressure) to drive the agent into the vasa vasorum. In some examples, one or more active agents for modulating the tone of the vasa vasorum may be included. For example, an arterial segment having sufficient vasa vasorum may be isolated proximate to the target region and a localized therapeutically effective dose of an agent (e.g., chemotherapeutic agent) may be delivered into the isolated region. Note that the fluid pressure within the occluded region results in a radial pressure against the wall (radially against the wall) of the vessel, rather than pressure applied as the fluid is moving through the vessel. Thus, the pressure may be referred to as static (hydrostatic) fluid pressure, and is distinct from pressure due to blood flow (e.g., blood pressure) within the vessel.

A region of a vessel may be sufficiently close to the target region when it is within a distance sufficient to allow delivery of the agent through the vasa vasorum connected to the vessel to reach target tissue within about 10 minutes (or sooner) after delivery. This may be confirmed by imaging (e.g., use of a dye or contrast agent). In some cases the target tissue may be within about 5 cm (within about 4 cm, within about 3 cm, within about 2 cm, within about 1 cm, within about 9 mm, within about 8 mm, within about 7 mm, within about 5 mm, etc.). In practice, the region of the vessel (e.g., artery) that may be adjacent to the target region, e.g., within about 5 cm, within about 4 cm, within about 3 cm, within about 2 cm, within about 1 cm, within about 9 mm, etc.).

A sufficient density of vasa vasorum may refer to the density of microvascularity around the vessel, and may be, e.g., 20% or more vascularized by microvascularity (e.g., 25% or more, 30% or more, 35% or more, 40% or more, 45% or more, 50% or more, 55% or more, 60% or more, etc. vascularized by microvascularity). Microvascularity may be determined and/or confirmed as described herein. In general, the microvascularity of a vessel or region of a vessel may vary based on the size and location of the vessel and may be characteristic of the region of the vessel.

The methods described herein may be particularly well suited to delivery of one or more agents through the vasa vasorum in which the size of the agent is larger and/or is charged or otherwise considered “sticky” and therefore believed to be a poor candidate for delivery through an otherwise intact blood vessel wall. For example, described herein are methods of delivering a virus (e.g., oncolytic virus, AAV, etc.) a nanoparticle, antibodies, cell and/or a small molecule. In particular, these agents may be charged. The agent may be larger than 100 nm in diameter (e.g., larger than 200 nm, larger than 300 nm, larger than 400 nm, larger than 0.5 μm, larger than 0.7 μm, larger than 0.8 μm, larger than 0.9 μm, larger than 1 μm, larger than 1.1 μm, larger than 1.5 μm, larger than 2 μm, larger than 2.5 μm, larger than 3 μm, larger than 5 μm, etc.). In general, the agent(s) may be any therapeutic agent.

For example, the agent may be a virus, such as but not limited to viruses from families varying in sizes and structures, from icosahedral to helical symmetry, with or without lipid envelope, integument or matrix and with variable susceptibility to physical disruption). In some examples, the agent is an adenovirus, herpes simplex virus, parvovirus, vaccinia virus, measles virus, Newcastle disease virus, reovirus, coxsackie virus, Seneca valley virus, poliovirus, vesicular stomatitis virus, pox virus.

In any of these methods and apparatuses, the agent may be a nanoparticle less than the size of the vasa vasorum. The agent may be a cell therapy.

The target tissue may be any appropriate target tissue, including in particular, target tissue including or adjacent vasa vasorum. For example, the target tissue may be the pancreas.

In some examples, the agent may be a gene therapy. The agent may be a nanoparticle including one or more polynucleotides configured to result in a gene therapy. In some examples the agent may be a therapeutic mRNA (which may be included with a nanoparticle.

In some examples the agent may be an antibody. For example the agent may be an antibody and an immune activator and/or a gene therapy and/or a therapeutic mRNA. In any of these examples the agent may include an immune activator (e.g., IL-12, IL-2, IL-15, TLR9 agonist, TLR7/8 agonist).

For example, described herein are methods comprising: identifying a region of a vessel that is proximate to a target region and a density of vasa vasorum having an average diameter size of greater than 10 μm (e.g., 12 μm, 15 μm, 17 μm, 19 μm, 20 μm, 22 μm, 25 μm, 27 μm, 30 μm, etc.) so as to deliver a target agent; occluding the identified region of the vessel, delivering the agent within the occluded region under pressure to drive the agent into the vasa vasorum, wherein the pressure (e.g., fluid pressure) is maintained at 150 mmHg or less (e.g., 140 mmHg or less, 130 mmHg or less, 120 mmHg or less, 110 mmHg or less, 100 mmHg or less, 90mmHg or less, 85 mmHg or less, 80 mmHg or less, 75 mmHg or less, 70 mmHg or less, 65mmHg or less, 60 mmHg or less, 55 mmHg or less, 50 mmHg or less, 45 mmHg or less, 40mmHg or less, 35 mmHg or less, 30 mmHg or less, 25 mmHg or less, etc.).

This patent application may be related to one or more of: U.S. patent application Ser. No. 17/367,046, titled “METHODS FOR TREATING CANCEROUS TUMORS, filed Jul. 2, 2021, U.S. patent application Ser. No. 17/315,220, titled “METHODS AND APPARATUSES FOR TREATING TUMORS,” filed May 7, 2021, U.S. patent application Ser. No. 16/685,974, filed Nov. 15, 2019, titled “METHODS FOR TREATING CANCEROUS TUMORS,” now U.S. Pat. No. 11,052,224, and U.S. patent application Ser. No. 15/807,011, titled “Methods for Treating Cancerous Tumors,” filed Nov. 8, 2017, the disclosure of each of which is incorporated herein by reference in its entirety.

For example, described herein are methods comprising: identifying a region of a vessel that is proximate to a target region and includes a sufficient density of vasa vasorum; isolating the identified region of the vessel by occluding an upstream region of the vessel and a downstream region of the vessel; and delivering an agent through the vasa vasorum by applying the agent within the occluded identified region while maintaining a fluid pressure within the isolated region. Delivering the agent may include delivering the agent concurrently with a vasodilator and/or shortly after delivering the vasodilator (e.g., within about 3 hours, 2 hours, 1 hour, 30 minutes, 20 minutes, 10 minutes, 5 minutes, etc.). The vasodilator may be nitroglycerine. In some examples delivering the agent may comprise delivering the agent within less than 3 hours after treating the target region with radiation. In general, identifying the region of the vessel comprises identifying a region that has a density of sufficiently sized diameter vasa vasorum, such as a density of vasa vasorum having an average size of greater than about 40 μm. The density and/or size may be estimated directly (by visualization) or indirectly, based on known or consensus properties of the vessels. For example, the region may be identified from a database or an anatomical atlas including typical densities and/or average sizes of vasa vasorum. In some examples identifying the region of a vessel that is proximate to the target region includes scanning with an imaging modality (e.g., optical coherent tomography, or OCT and/or CT, micro-CT, MRI, ultrasound, etc.). These methods and apparatuses may maintain a fluid pressure within the region between the occluders of, e.g., 150 mmHg or less (e.g., 140 mmHg or less, 130 mmHg or less, 120 mmHg or less, 110 mmHg or less, 100 mmHg or less, 90 mmHg or less, 80 mmHg or less, 70 mmHg or less, etc.). The fluid pressure may be maintained for 5 minutes or less (4.5 min or less, 4 min or less, 3.5 min or less, 3 min or less, 2.5 min or less, 2 min or less, 1.5 min or less, 1 min or less, etc.).

In general, the agent may be relatively large. For example, the agent may be larger than 500 nm in diameter. The agent may be a virus. In some examples the agent is a virus having one or more of: an icosahedral or helical symmetry, a lipid envelope, an integument or matrix, and a variable susceptibility to physical disruption. The agent may be one or more of: adenovirus, herpes simplex virus, parvovirus, vaccinia virus, measles virus, Newcastle disease virus, reovirus, coxsackie virus, Seneca valley virus, poliovirus, vesicular stomatitis virus and pox virus. The agent may be a nanoparticle. The agent may be a cell or tissue (e.g., part of a cell therapy). The agent may be a gene therapy. The agent may be an antibody. The agent may be an immune activator (e.g., IL-12, IL-2, IL-15, TLR9 agonist, TLR7/8 agonist).

For example, a method for delivering an agent through vasa vasorum may include: identifying a region of a vessel that is proximate to a target region, wherein the region comprises vasa vasorum; isolating the identified region of the vessel by occluding both an upstream and a downstream region from the identified region; and delivering the agent through the vasa vasorum by applying the agent within the isolated region, wherein a fluid pressure within the isolated region is maintained.

In some examples, a method for delivering an agent through vasa vasorum may include: identifying a region of a vessel that is proximate to a target region, wherein the region comprises vasa vasorum; isolating the identified region of the vessel by occluding both an upstream and a downstream region from the identified region; and delivering the agent through the vasa vasorum while the vasa vasorum is dilated by a vasodilator by applying the agent within the isolated region, wherein a fluid pressure within the isolated region is maintained.

In some examples a method (e.g., a method of delivering an agent through the vasa vasorum) may include: identifying a region of a vessel that is proximate to a target region and that has a density of vasa vasorum having an average diameter size of 10 μm or greater; isolating the identified region of the vessel by occluding an upstream region and a downstream region from the identified region; and delivering an agent within the isolated identified region under pressure to drive the agent into the vasa vasorum, wherein the pressure is maintained at 150 mmHg or less (e.g., 140 mmHg or less, 130 mmHg or less, 120 mmHg or less, 110 mmHg or less, 100 mmHg or less, etc.) Also described herein are systems for performing any of these methods. For example, described herein are systems for applying an agent through a vasa vasorum. The system may include: a catheter body comprising a first expandable occluder and a second expandable occluder arranged in series; an outlet positioned between the first expandable occluder and the second expandable occluder, wherein the outlet is fluid communication with a fluid line extending proximally down the catheter body to a proximal port; a pressure sensor configured to sense pressure in a region outside of the catheter body between the first expandable occluder and the second expandable occluder; and an imaging sensor configured to detect vasa vasorum at a distal end region of the catheter body.

The imaging sensor may comprise an optical coherence tomography (OCT) sensor, and/or an ultrasound sensor. In any of these examples the system may include on or more processors (e.g., including hardware such as a circuitry) coupled to the imaging sensor and configured to detect vas vasorum from the imaging sensor and to output an indicator of the presence of vasa vasorum at the distal end region of the catheter body. A processor includes hardware that runs the computer program code. Specifically, the term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices. The imaging sensor may be positioned between the first expandable occluder and the second expandable occluder. In some examples the imaging sensor is on either or both the first expandable occluder and the second expandable occluder. The first expandable occluder may comprise a first expandable balloon and the second expandable occluder comprises a second expandable balloon.

Any of these apparatuses may include a controller configured to apply an agent out of the outlet for delivery through the vasa vasorum by coordinating expansion of the first expandable occluder and the second expandable occluder after the presence of vasa vasorum is confirmed using the imaging sensor. The controller may be configured to maintain a fluid pressure between the first expandable occluder and the second expandable occluder while delivering the agent out of the outlet. The controller may be configured to maintain the fluid pressure of 150 mmHg or less (e.g., 140 mmHg or less, 130 mmHg or less, 120 mmHg or less, 110 mmHg or less, 100 mmHg or less, 90 mmHg or less, etc.). The controller may be configured to maintain the fluid pressure for 10 minutes or less (9 min or less, 8 min or less, 7 min or less, 6 min or less, 5 min or less, 4.5 min or less, 4 min or less, 3.5 min or less, 3 min or less, 2.5 min or less, 2 min or less, 1.5 min or less, 1 min or less, etc.).

In any of these systems the system may be configured for use with a vasodilator. For example, the system may include a reservoir configured to hold (and/or holding) a vasodilator fluidly, that is configured to be coupled to the fluid line. The reservoir may be, for example, a reservoir of nitroglycerine.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is an illustration of a catheter device disposed within a vessel, according to an embodiment.

FIG. 2 is a photograph showing blue dye infused from an internal lumen to vasa vasorum vessels when the lumen is occluded and dye is infused intraluminally across the vessel wall (e.g., by TAMP)

FIG. 3A is an electron micrograph showing small, scattered gold nanoparticles around the collagen fibrils of media. FIGS. 3B and 3C are electron micrographs showing gold nanoparticles in the perivascular space of adventitia. FIG. 3D is an electron micrograph showing gold nanoparticles within a large vessel of vasa vasorum of adventitia.

FIGS. 4A-4B illustrate examples of a treated tissue (e.g., porcine tissue) and the imaging results when delivering material (e.g., dye, drug, etc.) through the vasa vasorum as described herein.

FIGS. 5A-5B illustrate one example of a test tissue (e.g., porcine splenic tissue) into which material was delivered via the vasa vasorum from an artery. FIG. 5A shows a visible light image of the tissue. FIG. 5B shows an X-ray microCT image showing contrast delivered from the artery and through the vasa vasorum following treatment.

FIGS. 6A-6B illustrate an example of a test tissue (e.g., porcine splenic tissue) into which material was delivered from an artery without driving through the vasa vasorum as described herein. FIG. 6A shows a visible light image of the tissue. FIG. 6B shows an X-ray microCT image showing contrast was delivered only into the artery and not through the vasa vasorum.

FIG. 7 schematically illustrates one example of a method of delivering an agent through the vasa vasorum.

FIG. 8 schematically one example of a system for delivering an agent through the vasa vasorum.

### DETAILED DESCRIPTION

The methods and apparatuses described herein may be configured to use the vasa vasorum to deliver one or more agents into a target region of the adventitia in a manner that was previously not believed to be possible. The methods and apparatuses described herein may generally occlude a segment of a vessel (and in particular a vessel having a significant amount of vasa vasorum), increase the static pressure in the occluded segment to drive drug circumferentially out of the occluded segment and into surrounding tissue through the vasa vasorum.

In general, the methods and apparatuses described herein may include selectively occluding a region of a vessel, such as an artery, that includes (and in some cases is sufficiently dense in) vasa vasorum and applying one or more agents (e.g., drugs, cells, etc.) into the occluded region of the vessel so that the agent is taken up by the vasa vasorum in a manner that does not harm or occlude the vasa vasorum, to treat the target region with the agent. In some examples the size of the agent may be matched to the size of the vasa vasorum, and/or the region of the vessel including vasa vasorum that is matched to the size of the target (e.g., maximum size, average size, etc.). Any of these methods and apparatuses may be configured to confirm the presence, density, and/or size of the vasa vasorum. For example, any of these methods and apparatuses may include imaging of the vasa vasorum using an imaging modality that is appropriate. For example, any of these methods and apparatuses may include contrast-enhanced imaging modalities (e.g., magnetic resonance and CT) that may identify vasa vasorum. Micro-CT, optical coherence tomography (OCT), and magnetic resonance imaging (MRI) are high-resolution imaging modalities that may successfully detect vasa vasorum. Doppler and contrast-enhanced ultrasound (CEU) may be used; high-frequency intravascular ultrasound and/or super-resolution ultrasound (SRU) may also be used.

Blood vessels are composed of three distinct layers: the intima (inner, abluminal layer consisting of endothelial cells), the media (middle, smooth muscle, and elastic layer), and the adventitia (outer, fibrous layer). The vasa vasorum are a network of microvessels within larger arteries and veins with importance to the overall health of the larger “host” vessel. There exist arterial vasa vasorum to deliver oxygen and nutrients to the parent vessel and venous and lymphatic vasa vasorum, which remove waste and extracellular fluid to adjacent veins and lymphatic vessels, respectively. Two types of vasa vasorum (e.g., externa and interna) are described, characterized on the basis of their anatomical origin.

The distribution and density of vasa vasorum is not uniform. Some regions of the body (e.g., some arteries) include a greater density of vasa vasorum than other regions. The methods described herein may therefore confirm that the vessel into which the method is to be performed confirm the density of the vasa vasorum and/or any of these apparatuses described herein may be configured to confirm the density. Further, any of these methods and apparatuses may be configured to minimize any damage to the vasa vasorum and may be performed in a manner that limits damage to the vasa vasorum.

For example, the vasa vasorum may be more densely distributed in the ascending thoracic aorta, the brachiocephalic and coronary arteries. Intercostal arteries feed the vasa vasorum of the descending thoracic aorta, which are as similarly dense as the ascending thoracic aorta. Vasa vasorum may be less dense in the infrarenal abdominal aorta than in the thoracic aorta, and may not penetrate the medial layer as in the thoracic aorta. Only the basilar and vertebral intracranial arteries are believed to contain vasa vasorum.

In general, the density of vasa vasorum also varies by anatomic location within different vascular beds. Arterial vasa vasorum density varies across different regions of human aorta, with maximum density in the aortic arch decreasing distally to reach a minimum in the infrarenal abdominal aorta. Denser lymphatic vasa vasorum within the infrarenal abdominal aorta. Coronary arteries may have a higher density of vasa vasorum when compared to femoral and renal arteries, followed by carotid, femoral, and renal arteries, with random intravessel distribution in all vessels examined. Vasa vasorum exist in the extracranial vessels but are rare among intracranial vessels.

Vasa vasorum are endarteries, meaning that they are vessels that do not connect with neighboring arteries and are responsible for oxygenating the local tissue. A range of lumen diameters have been reported for various normal arteries and veins of animal models and human specimens. In some examples, vasa vasorum of less than 50 μm in diameter may be present in the aorta and pulmonary artery. Vasa vasorum lumen diameters in human great saphenous vein may range from 11 to ~36 μm. A range of sizes for vasa vasorum of human femoral, abdominal aorta, iliac, and renal arteries and human carotid artery have been reported, e.g., for different ranges of lumen diameters as the mean of all vessels measured for a given specimen: small (<50 um), mid-size (50 to 100 μm), and large (>100 μm). The mean ±SD for all patient specimens examined may be 40.0±15.5 μm with approximately three quarters of all vessels measuring <50 μm (72.2±17.5%).

The vasa vasorum network within parent arteries and veins follows a similar hierarchy as artery-arteriole-capillary where vessels oriented along the longitudinal axis of the host vessel are known as “first-order” and are similar in size to arterioles. “Second-order” vessels in capillary size range branch from first-order vessels and extend circumferentially around the host vessel. First-and second-order vasa vasorum contain smooth muscle cell layer(s), while smaller second-order vessels (<25 μm) vasa vasorum exhibit pericyte coverage with a-smooth muscle expression in subsets. Vasa vasorum are also able to regulate their own tone, as they are vasoresponsive to physiologic and neural stimuli.

In any of the methods and apparatuses described herein, one or more vasoactive agent may be included to modulate the vasa vasorum (e.g., retract or constrict, and/or prune or grow additional vasa vasorum), including changing the tone of the vasa vasorum. For example, FGF receptor 2—and VEGFR2 (or other proangiogenic factors) may govern the expansion of the vasa vasorum; pro-and/or antiangiogenic factors may be used. Any of the methods and apparatuses described herein may include an agent to enhance delivery through the vasa vasorum, such as nitroglycerine. A vasoactive agent, including but not limited to nitroglycerine may be delivered locally after occluding the vessel as described herein. For example, between about 0.01 mg and 5 mg of nitroglycerin may be given in a targeted manner prior or concurrent with delivery of one or more agents into the occluded region of the vessel.

In general, the methods and apparatuses described herein may treat or ameliorate a disease such as, but not limited to, solid cancerous tumors. In some examples, unlike previous methods and apparatuses, the treatment may be delivered into an intact (non-disrupted) microvasculature, such as the vasa vasorum. For example, these methods may be performed without disrupting the microvasculature by radiation. Intra-arterial delivery of an agent, including but not limited to a chemotherapy, may be an effective and safe in treatment of solid tumors.

For example, as part of the treatments described herein, the proximal and the distal part of the vasculature (e.g., an artery) closest to the tumor may be isolated using a double balloon catheter. Both the side and the terminal branches may be excluded, which prevents drug washout. The vasa vasorum in the area may therefore take up the agent. Upon expansion (e.g., inflation) of both expandable occluders/occlusive members (e.g., balloons) in the isolated arterial segment, the intra-luminal pressure is modified, such as by reducing to the level of interstitium (typically, 10-20 mmHg). A therapeutic agent such as, for example, a chemotherapeutic drug, can be infused into the isolated arterial segment. The infusion of the chemotherapeutic drug in the isolated region, without any major runoff, may increase in the intra-luminal pressure of at least about 30 mmHg in the isolated vessel segment. The pressure gradient forces the infused agent to traverse the arterial wall and enter the surrounding tissue, especially the vasa vasorum surrounding the vessel wall, with subsequent influx of the therapeutic agent into the tissue.

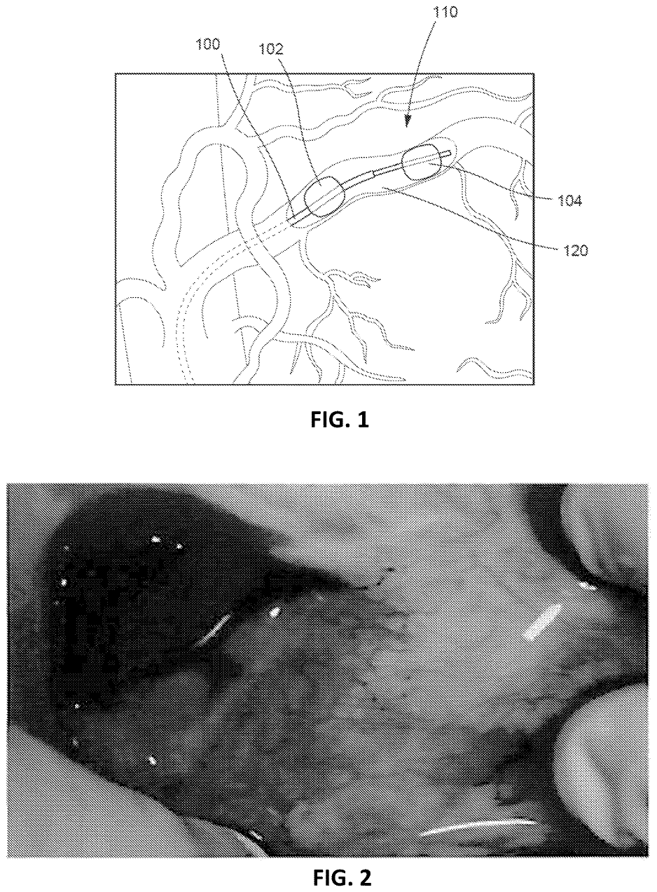

In some embodiments, catheter devices such as those described in U.S. patent application Ser. No. 14/293,603, filed Jun. 2, 2014, titled “Devices, methods and kits for delivery of therapeutic materials to a target artery,” now issued as U.S. Pat. No. 9,457,171, and U.S. patent application Ser. No. 14/958,428, filed Dec. 3, 2015, titled “Occlusion catheter system and methods of use,” the disclosures of which are incorporated herein by reference, can be used and/or adapted for use with the techniques described herein. FIG. 1 depicts an example catheter device 100. The catheter device 100 includes a first occlusion element 102 and a second occlusion element 104. The occlusion elements 102, 104 can be any suitable devices or mechanisms that are configured to selectively limit, block, obstruct, or otherwise occlude a bodily lumen (e.g., artery) in which the occlusion elements 102, 104 are disposed. For example, in some embodiments, the occlusion elements 102, 104 can be inflatable balloons or the like that can be transitioned between a collapsed (e.g., deflated) configuration and an expanded (e.g., inflated) configuration. The first occlusion element 102 can be coupled to a distal end portion of a first catheter, and the second occlusion element 104 can be coupled to the distal end portion of a second catheter. Alternatively, in some embodiments, the first occlusion element 102 and the second occlusion element 104 can be coupled to a single catheter at different points along the catheter. The catheter device 100 can be used to isolate a segment 120 of a bodily lumen (e.g., artery) within the space defined between the first occlusion element 102 and the second occlusion element 104. After the segment 120 is isolated, a procedure can be performed within the isolated segment 120 such as, for example, delivering a therapeutic agent to the isolated segment 120 and surrounding tissue 110.

While the catheter device 100 is depicted as having two occlusion elements 102, 104, other catheter devices that can be used with methods disclosed herein can include a single occlusion element or more than two occlusion elements (e.g., three occlusion elements), as needed to isolate a portion of a bodily lumen. For example, a catheter device with a single occlusion element can be used to isolate a portion of a vessel that is adjacent to or near a closed or terminating end of the vessel. Alternatively, a catheter device with three or more occlusion elements can be used to isolate a segment of a vessel that splits into one or more branches.

The methods and apparatuses described herein may tightly regulate the pressures applied, both to the occluders (e.g., balloons or other seals operating against the vessel wall) and between the occluders, exerting pressure to drive fluid into the vasa vasorum. This is because the vasa vasorum and vasa nervorum are particularly susceptible to external mechanical compression, and damage to these small vessels may result in disorders and tearing. Further, a tear in vasa vasorum may start pathologic cascade of events leading to aortic dissection. Thus, in general, it may be particularly important to regulate the pressure applied by these apparatuses both to form the seal (e.g., against the vessel walls) and between the sealed region, locally. In some examples the pressure acting against the vessels walls to “seal” may be particularly low, and may allow some leakage, rather than risk damage to the vasa vasorum. For example, the pressure applied by the seal (e.g., the occluders) against the wall may be less than x mmHg, where x is 10 mm Hg, 15 mmHg, 20 mmHg, 25 mmHg, 30 mmHg, 35 mmHg, 40 mmHg, 45 mmHg, 50 mmHg, 55 mmHg, 60 mmHg, 70 mmHg, 80 mmHg, 90 mmHg, 100 mmHg, 110 mmHg, 200 mmHg, 220 mmHg, 250 mmHg, 300 mmHg, 400 mmHg, 500 mmHg, 600 mmHg, 700 mmHg, 800 mmHg, 900 mmHg, 1000 mmHg, etc.

The pressure applied between the occluders may be limited to y mm Hg or less (e.g., where y may be 10 mm Hg, 15 mmHg, 20 mmHg, 25 mmHg, 30 mmHg, 35 mmHg, 40 mmHg, 45 mmHg, 50 mmHg, 55 mmHg, 60 mmHg, 70 mmHg, 80 mmHg, 90 mmHg, 100 mmHg, 110 mmHg, 200 mmHg, 220 mmHg, 250 mmHg, 300 mmHg, 400 mmHg, 500 mmHg, 600 mmHg, 700 mmHg, 800 mmHg, 900 mmHg, 1000 mmHg, etc.). In some cases the relative pressure applied between the occluders and the pressure of the sealing occluders against the vessel wall may generally be approximately the same or may be less.

Thus, any of these apparatuses may include one or more sensors for monitoring pressure within one or both occluder(s) (e.g., balloons) and/or applied by the occluders against the walls of the vessel and/or one or more pressure regulators regulating pressure applied by the fluid between the occluders.

### Delivery of Large Agents

The methods and apparatuses described herein may be useful to locally deliver agents that have not previously been possible to deliver through an intact vessel. It was previously believed that transport of materials through an intent blood vessel required passage through tight junctions, which limited both the size, amount and rate at which material could be delivered. This technique also limited to charge or “stickiness” of the material that could be delivered. In contrast, the methods described herein, which provide local isolation of a region of a vessel including vasa vasorum, may permit the passage of larger materials, including materials that have significant surface charge.

In some examples, therapeutic viruses (e.g., oncolytic viruses) and/or nanoparticles (including nanoparticles delivering therapeutic polynucleotides, such as therapeutic mRNA) may be locally and efficiently transported through a region of vasa vasorum as described herein. Oncolytic viruses often rely on intratumoral delivery to avoid immune detection in order to reach a tumor to replicate in and destroy cells, activating the immune system for subsequent systemic activation to distant sites. Unfortunately, intratumoral delivery is fraught with high variability in distribution. Additionally, pancreatic cancers have few tumor cells held deep within desmoplastic tissue. The methods described herein may be used to deliver oncolytic viruses.

In general, oncolytic viruses are also often given with immune checkpoint inhibitors, wherein tolerability has been a limiting issue due to pneumonitis, pancreatitis, and colitis. In previous studies, testing four various filters yielded a viral recovery below 25% for all four filters-highlighting the importance of membrane morphology in the yield and product recovery. Pressure has also been shown to be a pivotal factor in optimal oncolytic virus delivery, wherein tumors efficacy of oncolytic therapy is materially affected by the density and distribution of infectious centers within the tumor, which is likely influenced by the permeability and blood flow in tumor microvessels. In previous studies, researchers showed that treatment with higher perfusion pressure led to significant increase in survival compared to low perfusion. The techniques described herein may avoid these difficulties and permit passage of therapeutic viruses (e.g., oncolytic viruses) using the methods and apparatuses described herein.

### EXAMPLES

The methods and apparatuses described herein may force a drug through channels in the vessels “internal vas vasorum” that allow direct transfer of the agent (e.g., molecule/drug) across the artery wall to the adventitia. FIG. 2 illustrates an example of a blue dye that was infused from internal lumen to vasa vasorum vessels with occlusion of lumen and infusion of dye intraluminally across the vessel wall (e.g., by trans-arterial micro-perfusion or TAMP). As shown, the region stained blue illustrates the targeted distribution of the agent (indicated by the color).

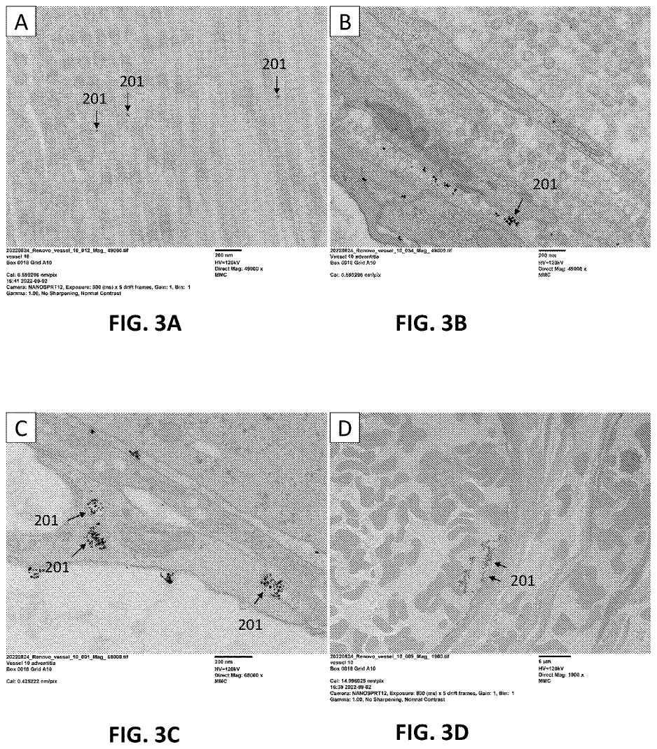

FIGS. 3A-3D show examples of electron microscopy images of infusion of 6 nm gold beads using the techniques described herein (e.g., TAMP) across arterial wall to vasa-vasorum with minimal penetration beads within the vascular media. This is evidence that the mechanism of transfer is direct connection to adventitia rather than diffusion via gap junctions across intima-media to adventia. In FIG. 3A the small, very few scattered gold nanoparticles 201 are shown around the collagen fibrils of media. In FIGS. 3B and 3C, the gold nanoparticles are shown in the perivascular space of adventitia. In FIG. 3D, gold nanoparticles are shown within a large vessel of vasa vasorum of adventitia. The distribution of particles within the vasa vasorum shown in FIGS. 3B, 3C and 3D, and the relative absence of particles within the adjacent tissue not part of the vasa vasorum illustrates that the occlusion and application of pressure as described herein specifically delivers the agent (e.g., particles) through the vasa vasorum, and is not a result of diffusion, which would result in distribution of the agent throughout the surrounding tissues.

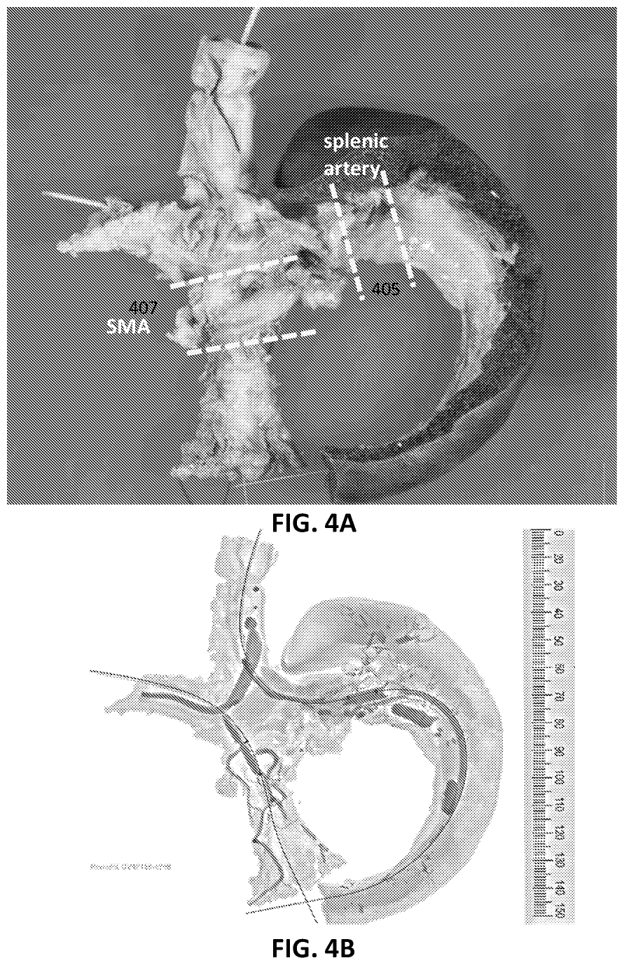

FIG. 4A illustrates an example showing a tissue having regions of vessels, e.g., splenic artery 505 and superior mesenteric artery (SMA) 507, into which an occluding device may be inserted to occlude a portion of these vessels (shown by dashed lines) for local delivery of a material (e.g., microfil contrast in this example) and delivery through the vasa vasorum as described. FIG. 4B shows an X-RAY Micro-CT scan through the whole tissue (e.g., using fluoroscopy). A double balloon catheter is introduced in splenic artery with isolation of arterial segment with inflated balloon and subsequent infusion of microfil contrast using TAMP (condition 1); in same animal another catheter was introduced into Superior Mesenteric artery (SMA), and micro-fil was injected under same condition without isolation of arterial segment (intra-arterial without TAMP-Condition 2).

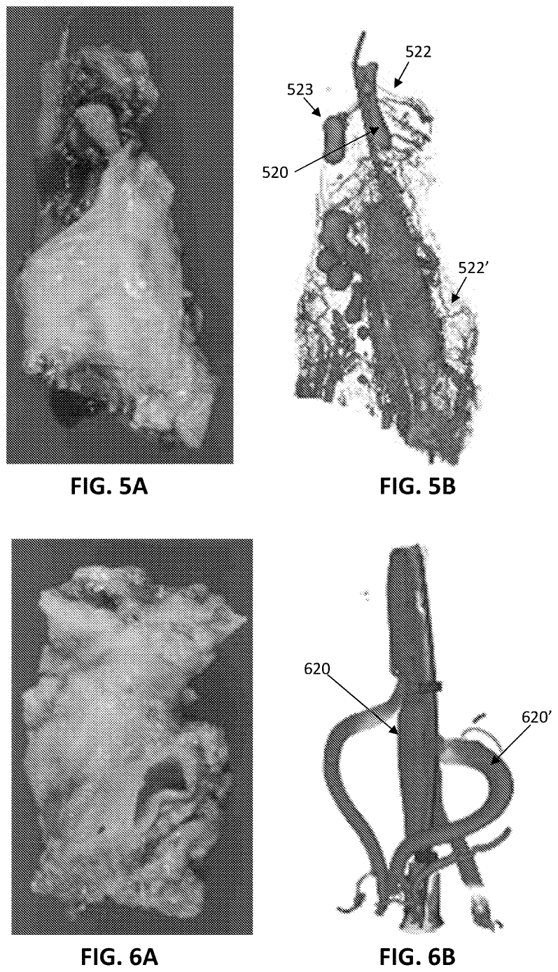

FIGS. 5A-5C illustrate an example of a region of tissue, in this case, as shown in FIG. 5A, shows resected section of tissue around splenic artery from FIG. 5; into which a device such as that shown in FIG. 1 is inserted in a region including a sufficient density of vasa vasorum. FIG. 5A shows the tissue region (from a porcine tissue model), while FIG. 5B shows a 3D rendered micro-CT images (5C) of the “splenic” artery 520 post injection of Microfil under condition 1. In this example, micro-fil dye (shown as a blue dye) of injection fluid was inserted into the tissue after occluding the region of the splenic artery, and imaged, as shown. The contrast material passed out of the vessels and through the vasa vasorum, providing a significant delivery, in a manner that is not diffusion limited, or dependent upon gap junctions, but instead is passed through the vasa vasorum and into the adjacent tissue regions. The filled vasa vasorum is indicated by the fine structures 522, 522′ and reservoir regions 522 labeled by the contrast material.

The example shown in FIGS. 5A-5C is very different from an example in which the vessel is not locally occluded, as shown in FIGS. 6A-6B. In this example a similar tissue region (FIGS. 6A-6B) may be treated, but without occluding both regions of the vessel and driving the material, in this example the contrast material, by injection through the vasa vasorum (condition 2). As shown in FIG. 6B, the contrast material is limited to the vessel bodies 620, 620′, but is substantially free from vasa vasorum and adjacent tissue.

In general, the methods and apparatuses described herein, by recruiting vasa vasorum interna (originating from intra-luminal space) may be used to deliver agent (e.g., drug) across the wall of the vessel and into the surrounding tissue. Although the vessels may not normally see a circumferential pressure, on the order used in the methods and apparatuses described for use herein, but instead see a longitudinal pressure (and flow), these methods and apparatuses may result in a targeted circumferential pressure (e.g., force) that recruits these micro-vessels to introduce the agent into the surrounding tissue. This was illustrated experimentally as described in FIGS. 3A-3D, 4A-4B, 5A-5B and 6A-6B, in which normal laminar flow of contrast in the artery resulted in little or no passage of agent through the vasa vasorum (see, e.g., FIG. 6B).

As mentioned, in any of the methods and apparatuses described herein, the apparatus may include an imaging sensor to confirm, quantify and/or monitor the vasa vasorum continuous with the vessel. For example, any of these apparatuses may include an optical sensor for performing optical coherence tomography (OCT) on the adjacent tissue. OCT may be used to image the vessel walls (with or without contrast) to detect or confirm the presence of intact vasa vasorum. In some examples the apparatus may include one or more ultrasound sensors for detecting vasa vasorum.

These methods and apparatuses may be configured to treat a tumor. For example, described herein are apparatuses (e.g., systems, devices, etc.) and methods for the treatment of tumors, including cancerous tumors.

In addition or instead of the use of one or more agents for local modulation of the vasa vasorum through a channel of the apparatuses (see, e.g., FIG. 1) described herein, any of these methods and apparatuses may be used with a treatment or therapy that generally modulates the microvasculature, such as a radiation therapy. Thus, a course of radiation therapy targeting an area including a solid tumor may be used, and may modulate the microvasculature in the area.

These methods and apparatuses may be used as described herein following the radiation therapy. For example, a dose of one or more chemotherapeutic agents may be delivered to an isolated arterial section near the solid tumor in which the arterial section includes vasa vasorum.

For example, these methods may include administering a targeted dose of radiation to an area including a solid tumor, and isolating an area containing a cancerous tumor by, for example, isolating an arterial segment proximate to the tumor; and administering a localized therapeutically effective dose of a chemotherapeutic agent through the vasa vasorum within a predetermined time period (e.g., before the microvasculature can be reduced). Because these methods and apparatuses may target the vasa vasorum, they may pass the drug or other agent actively, rather than rely on diffusion, e.g., through gap junctions, as previously believed. This may allow for significantly larger agents, as well as agents that are charged or uncharged (or some combination of charged and uncharged).

The method may include administering a course of radiation therapy to an area including a solid tumor; isolating the proximal and the distal part of the vasculature closest to the tumor to produce an isolated arterial segment; decreasing the intraluminal pressure of the isolated arterial segment to the level of the interstitium; and administering a therapeutically effective dose of a chemotherapeutic drug though the vasa vasorum.

In some examples the method includes delivering radiation therapy to a target area including a tumor; and inserting a catheter device into an artery where the catheter device includes a first occlusion member, a second occlusion member, and a body defining a lumen in fluid communication with an infusion port. The infusion port is disposed between the first occlusion member and the second occlusion member. The catheter may be positioned after targeting a vessel or vessel region that is enriched for vasa vasorum. The first occlusion member and the second occlusion member may be moved to an area of the artery disposed proximate to the target area while sufficiently enriched for vasa vasorum. The first occlusion member and the second occlusion member may be deployed to isolate the area of the artery disposed proximate to the target area. A dose of chemotherapeutic agent may then be delivered to the isolated area of the artery via the lumen and the infusion port. The chemotherapeutic agent permeates to the target area including the tumor from the isolated area of the artery and through the vasa vasorum.

In some embodiments, the method includes administering a dose of radiation to a target area including a tumor; inserting a catheter device into a vessel, the catheter device including a first occluder and a second occluder; isolating a segment of the vessel proximate to the target area using the first occluder and the second occluder; and delivering a dose of an agent to the segment via the catheter device.

In some embodiments, the method includes administering a dose of radiation to a target area including a tumor; isolating a segment of the vessel proximate to the target area; adjusting an intraluminal pressure of the segment to a level of pressure of an interstitial space between the vessel and the target area; and delivering a dose of an agent to the segment via the catheter device.

In general, the methods described herein may pre-treat the tissue (including tumor tissue) with radiation to modify the microvasculature, which may limit or prevent washout of the applied chemotherapeutic(s). Alternatively, any of these methods may be used without first applying radiation, or may be performed sufficiently soon after (or concurrent with) the radiation so that the vasa vasorum is intact before applying therapy. The method may be performed while the vasa vasorum (e.g., microvasculature) is sufficiently intact. Any of these methods may further include blocking or otherwise excluding side branches of the vasculature at or around the target tissue. For example, one or more coils may be used to exclude a side branch. In some examples, a glue or sealant may be used.

The application of radiation may be local to the target tissue. For example, a local radiation catheter may be used. As mentioned, the radiation may be delivered concurrently with or after the delivery of the agent through the vasa vasorum.

Alternatively or additionally other micro-vasculature modification techniques may be used, including dilation of the microvasculature. For example, one or more drug agents may be used, including but not limited to nitroglycerine.

Following modulation of the function of the microvasculature in the target tissue (including the tissue of and/or surrounding a tumor), which may include delivering through the microvasculature before a reduction and/or inhibition occurs (e.g., following radiation treatment), the agent (e.g., chemotherapeutic agent) may be applied to the target tissue by using two or more occluders within a lumen (including but not limited to the vasculature, such as arterial vasculature and venous vasculature) in or adjacent to the target tissue, so that the chemotherapeutic agent may be applied locally, e.g., under controlled pressure, to the target tissue.

Virtually any tumor tissue may be treated as described herein. An apparatus including two or more occluders may be used in any appropriate lumen within or adjacent to the target tumor(s). These apparatuses may generally be referred to as catheter devices. For example, the methods described herein may include using an apparatuses including two or more occluders for delivery of the therapeutic agent (e.g., chemotherapeutic) agent(s) may be used in a target lumen comprising an artery such as, but not limited to: gastro-duodenal artery, pulmonary artery, proper hepatic or left or right hepatic artery, superior mesenteric artery, celiac artery, inferior vesical artery, middle rectal artery, internal pudendal artery, pulmonary artery (and its sub-branches), uterine artery, arteries of the bladder (e.g., superior vesical branch of the internal iliac artery, inferior vesical artery, vaginal artery, obturator and inferior gluteal arteries), mesenteric artery, iliac artery (and its sub-branches), and/or the internal carotid artery (and it's sub-branches). The methods described herein may also include using an apparatus including two or more occluders as described herein to deliver a therapeutic agent (e.g., a chemotherapeutic agent) in a target lumen such as, but not limited to: a vein, a bronchial lumen, a lumen of the digestive tract (esophagus, stomach, duodenum, small intestine, colon, rectum, etc.), a lumen of the bile duct (e.g., cholangio and pancreas), a urethra, a fallopian tubes, etc.

Any appropriate chemotherapeutic agent may be used, including, but not limited to small molecule chemotherapeutic agents, immunochemotherapeutic agents, stem cells, hormones, particles (nanoparticles, microparticles, etc.) and combinations of these. For example, the chemotherapeutic agent may include one or more (including combinations) of: Paclitaxel, Abraxane, Everolimus, Erlotinib Hydrochloride, Fluorouracil, Irinotecan Hydrochloride, Olaparib, Mitomycin, Irinotecan Hydrochloride Liposome, Sunitinib Malate, Lanreotide Acetate, and Lutetium Lu 177-Dotatate. Examples of combinations include, but not limited to: Folfirinox (Leucovorin Calcium {Folinic Acic}-Fluorouracil- Irinotecan Hydrochloride-Oxaliplatin), Gemcitabine-Cisplatin, Gemcitabine-Oxaliplatin, and OFF (Oxaliplatin-Fluorouracil-Leucovorin Calcium {Folinic Acic}). Other chemotherapeutic agents may include one or more (including combinations) of: alkylating agents, Nitrosoureas, Antimetabolites, Anti-tumor antibiotics, Topoisomerase Inhibitors, Mitotic Inhibitors, Corticosteroids, All-trans-retinoic acid, Arsenic trioxide, Asparaginase, Eribulin, Hydroxyurea, Ixabepilone, Mitotane, Omacetaxine, Pegaspargase, Procarbazine, Romidepsin, Vorinostat, All-trans-retinoic acid, Cisplatin, Entrectinib, Larotrectinib Sulfate, Nitrosourea, Pembrolizumab, Temozolomide, Carmustine, Bevacizumab, Naxitamab, and Lomustine.

Other chemotherapeutic agents may include one or more (including combinations) of: tumor antigen, immunotherapy agents, immunomodulators (e.g., thalidomide, lenalidomide, pomalidomide, etc.), stem cells, radiotherapy particles, steroids, hormones, coagulants, sclerosing agents (e.g., doxycycline, thiotepa, bleomycin, minocycline, 5-fluorouracil, etc.), cross-linking agents, etc.

Any of the agents described above may be used in combination with each other and/or in combination with a contrast media for fluoroscopic visualization.

In practice, the methods described herein may include isolating the lumen within or immediately adjacent to the target tissue using any of the apparatuses described herein. These apparatuses may generally include two (or in some examples, more, such as three, four, etc.) occluders that may occlude the lumen to prevent flow in/out of the lumen and allow the local control of pressure within the lumen by applying material, such as fluid and/or agent (e.g., chemotherapeutic agent), into the portion of the lumen blocked off by the two or more occluders. The occluders may be adjustable, including the spacing between the occluders.

In one non-limiting example, the method may include isolating the lumen segment, such as an arterial segment, with a pair of occludes (such as, in one example a pair of balloon occluders) and adjusting the length between the occluders. The isolated segment (e.g., the isolated arterial segment) may then be filled with fluid including the agent at a controlled pressure for a controlled period of time, to deliver the agent into the target tissue through the vasa vasorum, before the microvasculature has been inhibited.

Alternatively or additionally, in some examples described herein, the apparatus for use with any of these methods may include a fixed distance between the two or more occluders. The apparatus may be chosen from a variety of apparatuses each having a specified length between the two (or more) occluders. The user (e.g., physician) may select the appropriate size based on the anatomy, which may be visualized prior to or during the procedure using CT scan ultrasonography, fluoroscopy, MRI, x-ray, or other imaging means known in the art.

In general, an occluder is an expandable structure (frame, balloon, etc.) that seals off the lumen to prevent flow of fluid past the occluder within the lumen, when the occluder is deployed. The occluder may have a deployed configuration which is expanded to occlude the lumen (and seal it at one site) and a delivery configuration in which the occluder is collapsed to a smaller profile. Examples of occluders include, but are not limited to balloons, μmbrellas, expandable frames or meshes that may support a sealing membrane, etc. For example, an occluder may be configured as an expandable parachute structure and/or as an expandable umbrella. In some examples the occluder is an expandable stent having one or more membranes within the stent body that prevent the flow of material (fluid, such as blood, etc.) through the expanded stent (fully or partially covered with an impermeable or semi-permeable coating) once deployed. In some cases, the occluder includes an expandable (e.g., nitinol, stainless steel, etc.) frame that supports a sealing membrane. The sealing membrane may be a polymeric material.

Any of these apparatuses (and methods of using them) may include pressure monitoring. In particular, the pressure between the occluders may be monitored. Pressure monitoring may include in-line monitoring using one or more pressure sensors positioned on the handle of the apparatus, in fluid communication with one or more openings into the region between the occluders that can therefore detect pressure within the isolated region of the lumen. One or more additional pressure sensors may be used to determine the pressure within all or some of the occluders, particularly in occludes that are expanded by fluid pressure. The fluid pressure within the isolated region of the lumen may be estimated (e.g., using a controller of the apparatus), and/or may be displayed, stored, transmitted, including wirelessly transmitted, and/or may be used as feedback to control the pressure within the isolated region of the lumen. For example, the pressure of the isolated region may be maintained within a predetermined range by, e.g., adding and/or removing fluid, including any of the agents described herein, from one or more openings in the apparatus between the occluders.

Any of the apparatuses described herein may include a lumen for a wire (e.g., guidewire) so that the apparatus may be delivered over the guidewire. The wire lumen may include a lubricious material, such as a coating or sleeve of lubricious material, etc. For example, any of these apparatuses may include a lubricious liner in the wire lumen. In some examples the apparatus may be configured as a rapid exchange and/or monorail apparatus, including a rapid exchange wire channel region at the distal end region of the apparatus. The rapid exchange region may be distal to the occluders, or it may span the occluders.

These apparatuses may include one or more structural reinforcements, such as braids, coils, etc. on all or a portion of the apparatus. For example, the occluders may include a reinforced region. The catheter forming the apparatus may be reinforced (e.g., the catheter extrusion may be a reinforced catheter extrusion, including a braid, coil, etc.).

Any of these apparatuses may include one or more markers for visualizing the position of the apparatus within the body. For example, the apparatus may include one or more radiopaque markers for visualizing the apparatus during insertion, operation and/or removal of the apparatus. In some examples the one or more radiopaque markers may be positioned on or adjacent to each of the occluders, which may provide an indication of the position and span of the isolated region of the lumen. One or more markers may be positioned outside of this region (e.g., a third marker may be fixed proximal to the proximal occluder). The marker may include markers for any appropriate visualization, including radiopaque (e.g., fluoroscopic imaging), ultrasound markers (ultrasonic imaging), etc.

In some examples, the method of isolating the region of the lumen may include a method of isolating a region of the lumen including or adjacent to a bifurcation. For example, in some examples the method may include expanding an occluder within a bifurcated region of the lumen, an occluder sufficiently conformal to occlude at a bifurcation, a bifurcated occluder that can individually occlude each branch of a bifurcation, or two or more independent occluders to occlude each branch of a bifurcation (and other branch vessels, as needed).

Other objects of the invention may be apparent to one skilled in the art upon reading the following specification and claims. All of the methods and apparatuses described herein, in any combination, are herein contemplated and can be used to achieve the benefits as described herein.

This disclosure is not limited to particular methodologies, or the specific compositions described. It is also understood that the terminology used herein is for the purpose of describing particular embodiments only, and is not intended to be limiting, since the scope of the present application will be limited only by the appended claims and their equivalents.

Unless defined otherwise, technical and scientific terms used herein have the same meaning as commonly understood by one of ordinary skill in the art to which this invention belongs. Although any methods and materials similar or equivalent to those described herein can be used in the practice or testing of the present application, the preferred methods and materials are now described.

As used herein and in the appended claims, the singular forms “a,” “and,” and “the,” include plural referents unless the context clearly dictates otherwise. Thus, for example, the term “chemotherapeutic” is intended to mean a single chemotherapeutic or a combination of chemotherapeutics; “a course of radiation therapy” is intended to mean one or more courses of radiation therapies, or combinations thereof; the term “agent” is intended to mean a single agent or a combination of agents, and so on and so forth.

As utilized in accordance with the present disclosure, the following terms, unless otherwise indicated, shall be understood to have the following meanings:

The words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the implant end first inserted inside the patient's body would be the distal end of the implant, while the implant end to last enter the patient's body would be the proximal end of the implant.

“Treat”, “treating” and “treatment” of cancerous tumors refer to reducing the frequency of symptoms of cancer (including eliminating them entirely), avoiding the occurrence of cancer, and/or reducing the severity of symptoms of cancer.

“Therapeutically effective amount” and “therapeutically effective dose” means the amount or dosage of a compound that, when administered to a patient for treating cancerous tumors, is sufficient to effect such treatment. The “therapeutically effective amount” or “therapeutically effective dose” will vary depending on, for example, the compound, the size of the tumor, and the age, weight, etc., of the patient to be treated.

Also described herein are methods for treating or ameliorating solid tumors (including cancerous tumors), by applying a therapeutic agent through the isolated vessel region including vasa vasorum, without (or prior to) reducing, inhibiting or suppressing the microvasculature around one or more tumors or between a lumen of a body (e.g., artery, vein, or any other both lumen) and the tumor(s). In some cases, once the presence of the microvasculature (e.g., vasa vasorum) has been confirmed, a region (“segment”) of the lumen of the body may be isolated, e.g., by occluding the end of the segment, and applying one or more chemotherapeutic agent into the segment under pressure so that it may be effectively delivered to the one or more tumors, without disrupting the tissue unduly. In some cases the microvasculature may be treated with a vasodilator, such as (but not limited to) nitroglycerine, that is first and/or concurrently administered to an area including one or more tumors (“target tissue region”) and contains vasa vasorum. A therapeutically effective amount of an agent may be administered locally through the vasa vasorum to an isolated section of the lumen near the solid tumor. In some examples, the lumen may be an artery. Isolation of the section (e.g., arterial section) may be accomplished by isolating the proximal and the distal part of the vasculature closest to the tumor whereby the intraluminal pressure may then be decreased to the level of the interstitium (or in some cases higher). The therapeutically effective dose of the agent may then be administered via infusion through the vasa vasorum, from the vessel. Thus, in some examples, a combination of radiation therapy and administering of the agent through the vasa vasorum may have a synergistic clinical effect when combined.

Although intra-arterial delivery of chemotherapy, including TAC and TACE, has been shown to be effective and safe in treatment of certain solid tumors, a prerequisite for effective TAC or TACE is the selective engagement of nearby arterial vessels and, more commonly, the vessels feeding the tumor itself. The precise engagement of the feeding or branch vessel remains a major limitation for expanding the use of trans-arterial chemo-delivery (TAC) or trans-arterial chemo-embolization (TACE) in solid tumors, including but not limited to, pancreatic adenocarcinoma. The isolation of the artery supplying the tumor or the relevant tissue can be a technical challenge for a number of reasons, for example: a) there are organs with no dedicated single blood vessel supplying those specific organs; b) side and terminal branches of an artery can cause collateral flow to tissues and organs beyond the area of interest; and c) the tumor feeder vessels may be too small for detection by angiography; and d) the feeding branch/artery cannot be cannulated.

To address these problems, methods disclosed herein may involve administering a therapy following confirming that the microvasculature in the region of the tissue between an adjacent lumen and the tumor(s), is intact and/or sufficiently dense. After the therapy, the proximal and the distal part of the lumen (e.g., the vasculature such as an artery, vein, etc.) closest to the tumor(s) is isolated using an apparatus having two or more occluders, such as a double balloon catheter. Both the side and the terminal branches may be excluded, which prevents drug washout. The reduced microvasculature in the tissue in the area also reduces drug washout. Upon expanding the occluders, e.g., by inflation of both balloons, in the isolated luminal segment, the intra-luminal pressure may be increased above the level of interstitium (typically, about 10-20 mmHg). A therapeutic agent such as, for example, a chemotherapeutic drug, can be infused into the isolated arterial segment through the intact vasa vasorum. The infusion of the agent (e.g., chemotherapeutic drug) in the isolated region, without any major runoff, may lead to an increase in the intra-luminal pressure of at least about 30 mmHg in the isolated luminal segment. The pressure gradient may force the infused agent through the vasa vasorum surrounding the vessel wall, with subsequent influx of the therapeutic agent into the tissue. Although the method is not limited to arteries, but may be used with virtually any lumen, for convenience, this technique may be referred to as “trans-arterial micro-perfusion,” or TAMP, through the vasa vasorum.

The therapeutic agent (e.g., drug) may traverse the luminal wall (e.g., in arteries, endothelium and media) through the vasa vasorum. The interstitial concentration achieved may depend on the state of the vasa vasorum and the applied pressure, e.g., the intraluminal pressure achieved between the occluders (e.g., in some examples, balloons), the intraluminal drug concentration, and the duration of infusion. By varying these parameters, one can change the drug influx and interstitial concentration.

In some embodiments, catheter devices such as those described in U.S. patent application Ser. No. 14/293,603, filed Jun. 2, 2014, titled “Devices, methods and kits for delivery of therapeutic materials to a target artery,” now issued as U.S. Pat. No. 9,457,171, and U.S. patent application Ser. No. 14/958,428, filed Dec. 3, 2015, titled “Occlusion catheter system and methods of use,” the disclosures of which are incorporated herein by reference, can be used and/or adapted for use with the techniques described herein.

In combination with the techniques described above, if one can decrease the tissue efflux of the drug, the drug concentration near an isolated segment of a bodily lumen may be advantageously increased. When a tumor is located in this region, the increased concentration can increase the effect of the chemotherapeutic drug on the tumor.

Methods described herein can be used to treat solid cancerous tumors arising from any organ of the body where the tumor has its own or a proximate blood supply provided by a bodily lumen (e.g., artery) that can be isolated. Examples of cancers that can be treated using methods described herein can be, but are not limited to, pancreatic cancer, lung cancer, liver cancer, uterine cancer, colon cancer, or brain cancer.

For example, apparatuses and methods described herein can be used to isolate a targeted region in a patient's tissue to treat a tumor. Such tumors include, but are not limited to, pancreatic tumors, lung tumors, brain tumors, liver tumors, uterine tumors, and colon tumors. For example, when treating a lung rumor, these methods may include performing a method as described herein within the pulmonary artery. These methods may include performing the method in a gastro-duodenal artery. When treating the brain, the method may include optionally confirming that the internal carotid artery region to be treated includes sufficient vasa vasorum, and/or anterior and/or middle cerebral arteries. In some examples these methods may be performed in a vertebral artery.

In some embodiments, a method of treating a cancerous tumor can involve: first administering a course of radiation therapy targeting tissue including a solid cancerous tumor; second (or concurrently) treating to pass an agent though the vasa vasorum prior to the effects of the radiation being complete (e.g., the destructive effect of the radiation on the vasculature to take effect; and third administering a therapeutically effective dose of a chemotherapeutic agent to an isolated section of a bodily lumen near the solid tumor. The targeted solid tumor can be, for example, a pancreatic tumor, a lung tumor, a brain tumor, a liver tumor, a uterine tumor, a colon tumor, or virtually any other type of tumor. The administration of radiation on the targeted tissue area can include, for example, delivering approximately 20 to 50 Gy of radiation over approximately one to five weeks in approximately one to 25 sessions. The period of time between administration of the radiation therapy and administration of the chemotherapeutic agent can be selected to minimize the devascularization of the tissue surrounding the tumor. Depending on various factors including the specific course of radiation and the specific tissue area or organ, this period of time can be, for example, approximately within minutes hours or days. Examples of suitable chemotherapeutic agents include doxorubicin, erlotinib hydrochloride, everolimus, 5-FU, flurouracil, folfirinox, gemcitabine hydrochloride, gemcitabine-cisplatin, gemcitabine-oxaliplatin, irinotecan hydrochloride liposome, leucovorin, mitomycin C, mitozytrex, mutamycin, oxaliplatin, paclitaxel, paclitaxel albumin-stabilized nanoparticle formulation, or sunitinab malate or a combination of these drugs. In some embodiments, the section of the bodily lumen near the cancerous tumor can be isolated by the use of a catheter device to deliver the chemotherapeutic agent. In some embodiments, the catheter device can be used to increase the intraluminal pressure in the isolated section of the bodily lumen to achieve increased tissue penetration. Examples of suitable chemotherapeutic agents include doxorubicin, erlotinib hydrochloride, everolimus, 5-FU, flurouracil, folfirinox, gemcitabine hydrochloride, gemcitabine-cisplatin, gemcitabine-oxaliplatin, irinotecan hydrochloride liposome, leucovorin, mitomycin C, mitozytrex, mutamycin, oxaliplatin, paclitaxel, paclitaxel albumin-stabilized nanoparticle formulation, or sunitinab malate or a combination of these drugs.

In any of these methods and apparatuses, the surrounding area can be visualized to determine whether the vasa vasorum is intact and sufficiently dense. For example, the injection of contrast through the infusion port can also ensure that no extra vessels or bodily lumens are included in the isolated area. If desired, the catheter can be moved, and the procedure repeated until the clinician can confirm that the catheter is correctly positioned. After the positioning of the catheter is confirmed, a therapeutic cell/biologic/agent can be introduced to the isolated segment through the infusion port.

The use of radiation may otherwise reduce the microvasculature, so that when a drug is delivered to the area using methods described herein, it may be beneficial to perform the method prior to the effects of radiation reducing the microvasculature. After radiation treatment, the number of microvasculature connections (e.g., micro-vessels extending from the isolated section to the venous system.

### Radiation Therapy

In methods described herein, radiation therapy can include, for example, external-beam radiation therapy delivered by X-rays, gamma rays, proton beams, or other appropriate sources. Radiation therapy damages cells by destroying the genetic material that controls how cells grow and divide. While both healthy and cancerous cells are damaged by radiation therapy, the goal of radiation therapy is to destroy as few normal, healthy cells as possible. The radiation therapy described herein can be targeted as narrowly as possible to the solid tumor(s) being treated or the tissue closely surrounding the solid tumor(s).

Typically, a radiation treatment plan is individualized for a patient, based upon detailed imaging scans showing the location of a patient's tumor(s) and the normal areas around it. The amount of radiation that normal tissue in different parts of the body can safely receive is known to one skilled in the art. Computed tomography (CT) scans are most frequently employed, but magnetic resonance imaging (MRI), positron emission tomography (PET), and ultrasound scans may also be used. A radiation oncologist determines the exact area that will be treated, the total radiation dose that will be delivered to the tumor, how much dose will be allowed for the normal tissues around the tumor, and the safest angles (paths) for radiation delivery. Radiation doses for cancer treatment are measured in Gy, which is a measure of the amount of radiation energy absorbed by one kilogram of human tissue. Different doses of radiation are needed to kill different types of cancer cells. Patients can receive external-beam radiation therapy in daily treatment sessions over the course of several weeks. The number of treatment sessions depends on many factors, including the total radiation dose that will be given. For example, one dose, which constitutes a fraction of the total planned dose of radiation, can be given each day. In a different instance, two treatments a day can be given.

As will be appreciated by one skilled in the art, the course of radiation therapy appropriate for use in the method of the present invention will depend on the specific cancerous tumor being treated. The specific dose of radiation, the duration of the radiation, and the number of treatments for any particular individual will depend upon a variety of factors including the type of cancer, the size of the tumor(s), and the patient's age and medical history including, for example, the amount of radiation previously received. Concurrent chemotherapy may also impact the dose of radiation given.

When treating a pancreatic cancer, for example, the course of radiation therapy can be approximately 20 to 50 Gy of radiation delivered in approximately one to 25 treatments over approximately one to five weeks. Alternatively, two to five sessions of radiation can be given over a period of approximately a week. For certain types of cancer, the amount of radiation therapy delivered may be as low as one Gy. In preferred embodiments, the course of radiation therapy can be approximately 40 to 50 Gy of radiation delivered in approximately 22 to 25 treatments over approximately four to five weeks.

In methods described herein, after administering the radiation therapy, a physician concurrently or immediately deliver chemotherapy such that the tumorous tissue can die (e.g., necrosis) while the therapeutic agent is delivered through the intact or relatively intact vasa vasorum.

### Chemotherapeutics

Specific chemotherapeutics can be selected based on the particular solid tumor that is to be treated. For example, the following chemotherapeutic agents and others may be used in the treatment of pancreatic cancer: doxorubicin, erlotinib hydrochloride, everolimus, 5-FU, flurouracil, folfirinox, gemcitabine hydrochloride, gemcitabine-cisplatin, gemcitabine-oxaliplatin, irinotecan hydrochloride liposome, leucovorin, mitomycin C, mitozytrex, mutamycin, oxaliplatin, paclitaxel, paclitaxel albumin-stabilized nanoparticle formulation, or sunitinab malate. In some embodiments, a combination of agents may be employed. For example, when treating pancreatic cancer, a combination of gemcitabine hydrochloride (Gemzar®) and paclitaxel albumin-stabilized nanoparticle formulation (Abraxane®) may be used.

In general, any appropriate chemotherapeutic agent may be used, including, but not limited to small molecule chemotherapeutic agents, immunochemotherapeutic agents, stem cells, hormones, particles (nanoparticles, microparticles, etc.) and combinations of these. For example, in addition to those already described above, the chemotherapeutic agent may include one or more (including combinations) of: Paclitaxel, Abraxane, Everolimus, Erlotinib Hydrochloride, Fluorouracil, Irinotecan Hydrochloride, Olaparib, Mitomycin, Irinotecan Hydrochloride Liposome, Sunitinib Malate, Lanreotide Acetate, and Lutetium Lu 177-Dotatate. Examples of combinations include, but not limited to: Folfirinox (Leucovorin Calcium {Folinic Acic}-Fluorouracil-Irinotecan Hydrochloride-Oxaliplatin), Gemcitabine-Cisplatin, Gemcitabine-Oxaliplatin, and OFF (Oxaliplatin-Fluorouracil-Leucovorin Calcium {Folinic Acic}). Other chemotherapeutic agents may include one or more (including combinations) of: alkylating agents, Nitrosoureas, Antimetabolites, Anti-tumor antibiotics, Topoisomerase Inhibitors, Mitotic Inhibitors, Corticosteroids, All-trans-retinoic acid, Arsenic trioxide, Asparaginase, Eribulin, Hydroxyurea, Ixabepilone, Mitotane, Omacetaxine, Pegaspargase, Procarbazine, Romidepsin, Vorinostat, All-trans-retinoic acid, Cisplatin, Entrectinib, Larotrectinib Sulfate, Nitrosourea, Pembrolizumab, Temozolomide, Carmustine, Bevacizumab, Naxitamab, and Lomustine.

Other chemotherapeutic agents may include one or more (including combinations) of: tumor antigen, immunotherapy agents, immunomodulators (e.g., thalidomide, lenalidomide, pomalidomide, etc.), stem cells, radiotherapy particles, steroids, hormones, coagulants, sclerosing agents (e.g., doxycycline, thiotepa, bleomycin, minocycline, 5-fluorouracil, etc.), cross-linking agents, etc.

Any of the agents described above may be used in combination with each other and/or in combination with a contrast media for fluoroscopic visualization.

The above-described chemotherapeutic agents are available from a variety of sources licensed to provide such agents for human use. Generic formulations of non-proprietary chemotherapeutics are typically available from a variety of manufacturers. A list of these licensed suppliers is available from the U.S. Food and Drug Administration's “Approved Drug Products with Therapeutic Evaluations,” commonly known as the “Orange Book” (http://www.accessdata.fda.gov/scripts/cder/ob/). Proprietary chemotherapeutics are typically available from one manufacturer, also identifiable in the Orange Book. For example, the corporate source for Gemzar® is Eli Lilly and Company (Indianapolis, Ind.) and Celgene Corporation (Summit, N.J.) supplies Abraxane®.

Methods described herein can use an amount of chemotherapeutic agent that is known to be therapeutically effective at treating a tumor. For example, the amount of chemotherapeutic agent that is used can be based on the Prescribing Information for a particular chemotherapeutic drug. A physician can adjust the amount of the chemotherapeutic agent to an amount that is appropriate for use with the techniques described herein.

### Apparatuses

In some examples the apparatuses for use to perform the methods described herein may include catheter devices or systems. In some examples, the methods described herein can use a catheter device such as, for example, a device including two or more occluders, in some examples a double occlusion balloon device, to isolate a segment of a bodily lumen (e.g., artery) and allow infusion of a therapeutic agent (e.g., chemotherapy drug) into the isolated segment between the occluders after they are inflated. For example, methods disclosed herein may use catheter devices such as those described in U.S. patent application Ser. No. 14/293,603, filed Jun. 2, 2014, titled “Devices, methods and kits for delivery of therapeutic materials to a target artery,” now issued as U.S. Pat. No. 9,457,171, and U.S. patent application Ser. No. 14/958,428, filed Dec. 3, 2015, titled “Occlusion catheter system and methods of use,” which are incorporated herein by reference. Briefly, a catheter device suitable for isolating a section of a bodily lumen near a solid tumor includes, but is not limited to, features and functions such as, for example: (1) selective isolation of the targeted portion of the portion of the artery for targeted delivery of the therapeutic agent to the solid tumor; (2) an infusion port allowing first, injection of contrast into the isolated segment to allow direct visualization of the origin of the branches of the artery supplying the cancerous tissue, and second, introduction of chemotherapeutic drugs; and (3) a self-contained assembly unit with easy retrieval after completion of the procedure. In one embodiment, the catheter device includes expandable occluders in the form of inflatable balloons that can be used to isolate a proximal and distal end of a bodily lumen of interest.

As illustrated above, in some examples, the apparatus may be adjusted so that the distance between the occluders may be adjusted (increased or decreased).

Methods described herein can include, for example, introducing an apparatus (e.g., a catheter device) into a lumen such as into a splenic artery of the pancreas, or other appropriate body lumen adjacent and/or in a target tumor. The apparatus can have, for example, at least two lumens—one for inflation/deployment of the balloons/occluding elements and a second for introduction of the infusate (e.g., therapeutic agent) to the space between the two balloons. The catheter can be advanced to a target portion of the splenic artery. In some embodiments, the method can include advancing at least a portion of the catheter device to an ostium of a celiac artery, its hepatic branch (and its branches), or if necessary, the superior mesenteric artery, depending on a patient's anatomy. In some embodiments, a contrast dye is injected into the isolated region to confirm exclusion of side branches before injecting the infusate.

In some embodiments, the apparatus can have one or more features to achieve a desired effect on a specific anatomy of tumors. For example, there may be: (1) a separate inflation lumen for the proximal and the distal occluders/balloons to allow different size occluders/balloons proximally and distally; (2) slidable catheters to allow the distance between the occluders/balloons to be adjusted; and (3) a sensor at the tip to monitor pressure in the isolated segment of the bodily lumen.

In some embodiments, the catheter device can have a sensor such as a pressure transducer that may assist with achieving an optimal pressure within an occluded arterial segment for optimizing trans-arterial diffusion of an infused substance during a method of cancer treatment (e.g., a TAMP procedure). The pressure transducer may be disposed along the catheter device in the isolated arterial segment (e.g., disposed between the first occluder and the second occluder. The pressure transducer can be disposed on one of the catheters, or disposed on one of the occluders. The pressure transducer can be designed to measure an intraluminal pressure of the isolated segment. The pressure measurements may be used to adjust the intraluminal pressure of the isolated segment to a predetermined or optimal pressure level. A physician may use the pressure measurements to determine a rate of infusing a drug or other therapeutic material into the isolated segment in order to decrease or increase the intraluminal pressure of the isolated segment. For example, a physician can increase the rate of infusion of a drug to increase the intraluminal pressure of the isolated segment above the pressure of tissue surrounding the isolated segment (e.g., above the pressure of the interstitium) to create a pressure gradient between the intraluminal space and the surrounding tissue to increase permeation of the infused drug through the arterial wall and into the tissue. Additionally or alternatively, a physician can increase or decrease the intraluminal pressure of the isolated segment by adjusting the position of the two occluders relative to one another (e.g., moving the two occluders closer or further apart from one another). A sensor outside of the segment may be present distally or proximally.

Although the examples of the methods described above typically include accessing and isolating a portion of an artery, any of these methods and apparatuses may be used in other body lumen as well, including veins. In some cases it may be advantageous to access the target lumen through a shunt or graft that allows for easier re-access to the same target lumen for additional treatment cycles. Thus, aalternatively or additionally, these methods and apparatuses may be used in a patient having an arteriovenous (A-V) shunt, which may be used as described herein. In some cases, the shunt may be fitted to a patient and may be used for providing access for delivery of the treatment as provided hereinto to a region proximal or within the tumor.

In general, the methods described herein may be used in any target tissue in order to treat a tumor. Specifically, the apparatus including two or more occluders may be used in any appropriate lumen within or adjacent to the target tissue. For example, the methods described herein may include using an apparatuses including two or more occluders for delivery of the therapeutic agent (e.g., chemotherapeutic) agent(s) may be used in a target artery such as, but not limited to: gastro-duodenal artery, pulmonary artery, proper hepatic or left or right hepatic artery, superior mesenteric artery, celiac artery, inferior vesical artery, middle rectal artery, internal pudendal artery, pulmonary artery, uterine artery, arteries of the bladder (e.g., superior vesical branch of the internal iliac artery, inferior vesical artery, vaginal artery, obturator and inferior gluteal arteries), mesenteric artery, iliac artery (and its sub-branches), and/or the internal carotid artery (and it's sub-branches). The methods described herein may also include using an apparatus including two or more occluders as described herein to deliver a therapeutic agent (e.g., a chemotherapeutic agent) in a target lumen such as, but not limited to: veins (or in some examples, a shunt coupled to a vein), a bronchial lumen, a lumen of the digestive tract (esophagus, stomach, duodenum, small intestine, colon, rectum, etc.), a lumen of the bile duct (e.g., cholangio and pancreas), urethral, fallopian tubes, etc.

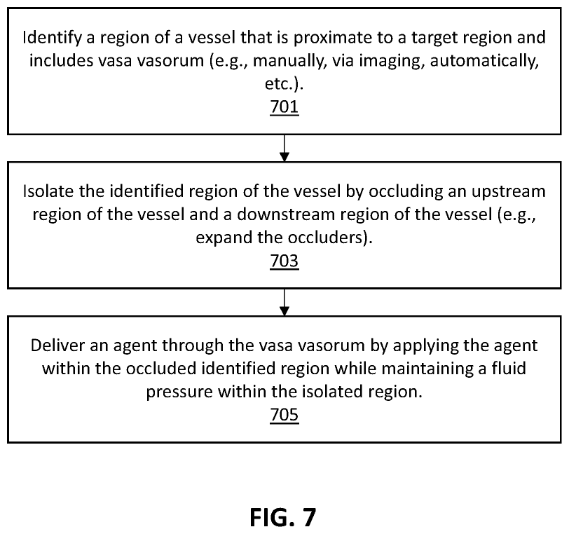

FIG. 7 schematically illustrates one example of a method of delivering an agent through the vasa vasorum. In this example, the method may include initially inserting a device (e.g., catheter) into a vessel near (e.g., adjacent) a target region. The method may include identifying a region of a vessel that is proximate to a target region and that also includes a sufficient density of vasa vasorum 701. As described above, the sufficient density may include a population of vasa vasorum having an average diameter of greater than about 10 μm (e.g., greater than about 20 μm, greater than about 30 μm, greater than about 40 μm, greater than about 50 μm, etc.). The density (e.g., presence, concentration, distribution, etc.) of the vasa vasorum may be measured and/or estimated. In some example the vasa vasorum may be detected using an imaging modality as described herein (e.g., OCT, ultrasound, etc.). In some examples the vasa vasorum may be estimated based on the location of the end region (occluding region) of the catheter. Once confirmed, the catheter may be positioned in the region of the vessel proximate to the target region having vasa vasorum, and the region may be isolated, e.g., the identified region of the vessel may be isolated by occluding an upstream region of the vessel and a downstream region of the vessel (e.g., expand the occluders) 703. Isolation may be configured by one or more sensors, including pressure sensor(s). Once isolated, the agent may be delivered through the vasa vasorum by applying the agent within the occluded identified region while maintaining a fluid pressure within the isolated region 705.

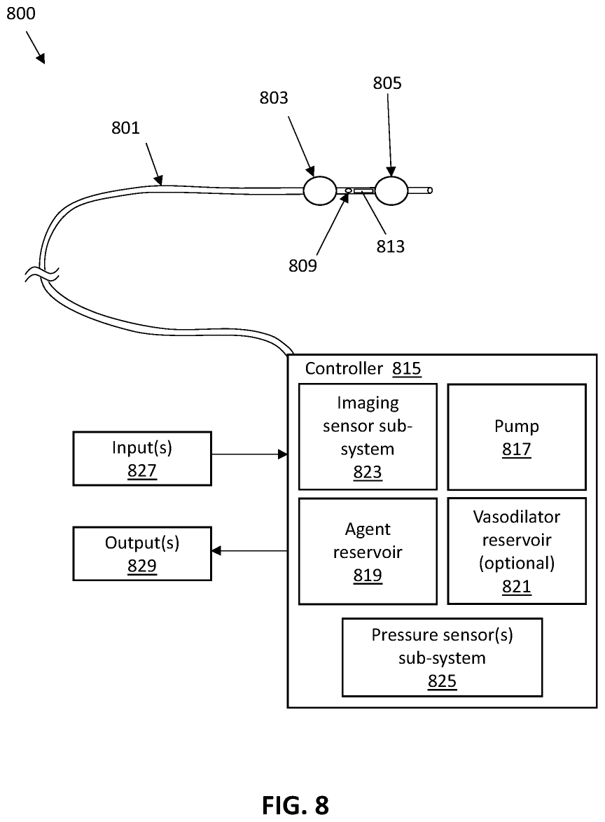

FIG. 8 illustrates one example of an apparatus (e.g., system) that is configured to deliver an agent (e.g., drug) through the vasa vasorum. In FIG. 8 the apparatus is shown as a system 800 that includes a catheter having a catheter body 801. The distal end region of the catheter includes a pair of expandable occluders 803, 805 (e.g., first occluder 803 and second occluder 805) that may be expanded or contracted (shown in the expanded configuration in FIG. 8) to occlude the vessel. An outlet 809 is positioned between the occluders and may fluidly connect to a fluid line extending through the catheter body. A proximal end of the catheter body may include an optional handle, and may couple to a control 815 including one or more processors. The fluid line may also connect to one or more reservoirs, such as an agent reservoir 819 and an optional dedicated vasodilator reservoir (821). The controller may control a pump 817 (e.g., peristaltic pump, syringe pump, etc.) that may drive, at a controlled flow and/or pressure, fluid (e.g., agent) into the region between the occluders 803, 805. The controller may receive input from one or more subsystems, including a pressure and/or flow sensing subsystem 825. The apparatus may optionally include an imaging sensor 813 and imaging sensor sub-system 823 that may be used to detect vasa vasorum. The imaging sensor may be any appropriate imaging sensor, including an optical sensor (e.g., OCT) sensor and sub-system, an ultrasound sensor and sub-system, etc. In FIG. 8 the imaging sensor is shown on in the region between the occluders, but in some cases it may be on the distal end region, e.g., distal to the occluders, and/or it may be on the occluders (to allow it to be driven against the vessel wall. IN some cases the imaging sensor may be part of an accessor device that is, e.g., inserted through a lumen of the catheter body.

In general, the system may include an input 827 for user control and operation of the system and output 829, which be, e.g., one or more LEDS, a display, touchscreen, audio output, etc. The input may include a touchscreen, one or more buttons, controls, knobs, dials, pedals, etc.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments as discussed above. For example, the size and specific shape of the various components can be different from the embodiments shown, while still providing the functions as described herein. Furthermore, each feature disclosed herein may be replaced by alternative features serving the same, equivalent or similar purpose, unless expressly stated otherwise. Thus, unless expressly stated otherwise, each feature disclosed is one example only of a generic series of equivalent or similar features.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, pressure, flow, temperature, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

1. A method comprising:
identifying a region of a vessel that is proximate to a target region and includes vasa vasorum;
isolating the identified region of the vessel by occluding an upstream region of the vessel and a downstream region of the vessel; and
delivering an agent through the vasa vasorum by applying the agent within the occluded identified region while maintaining a fluid pressure sufficient to force the agent across the arterial wall (through the vasa vasorum) within the isolated region.

2. The method of claim 1, wherein delivering the agent comprises delivering the agent concurrently with a vasodilator.

3. The method of claim 1, wherein delivering the agent comprises delivering the agent with nitroglycerine.

4. The method of claim 1, wherein delivering the agent comprises delivering the agent within less than 3 hours after treating the target region with radiation.

5. The method of claim 1, wherein the vessel is an artery.

6. The method of claim 1, wherein the target region comprises a tumor.

7. The method of claim 1, wherein identifying the region of the vessel comprises identifying a region that is includes vasa vasorum having an average diameter greater than about 40 μm.

8. The method of claim 1, wherein identifying the region of a vessel that is proximate to the target region and includes scanning with an imaging modality.

9. The method of claim 8, wherein the imaging modality comprises optical coherence tomography (OCT).

10. The method of claim 1, wherein the fluid pressure is maintained at less than 150 mmHg.

11. The method of claim 1, wherein the fluid pressure is maintained at less than 100 mmHg.

12. The method of claim 1, wherein the fluid pressure is maintained for 5 minutes or less.

13. The method of claim 1, wherein the agent is larger than 500 nm in diameter.

14. The method of claim 1, wherein the agent is a virus.

15.-18. (canceled)

19. The method of claim 1, wherein the target region is a region of pancreas.

20. The method of claim 1, wherein the agent is a gene therapy.

21. The method of claim 1, wherein the agent is an antibody.

22. The method of claim 1, wherein the agent is an immune activator (e.g., IL-12, IL-2, IL-15, TLR9 agonist, TLR7/8 agonist).

23. (canceled)

24. A method for delivering an agent through vasa vasorum, the method comprising:
identifying a region of a vessel that is proximate to a target region, wherein the region comprises vasa vasorum;
isolating the identified region of the vessel by occluding both an upstream and a downstream region from the identified region; and
delivering the agent through the vasa vasorum while the vasa vasorum is dilated by a vasodilator by applying the agent within the isolated region, wherein a fluid pressure within the isolated region is maintained.

25. A method comprising:
identifying a region of a vessel that is proximate to a target region and that has a density of vasa vasorum having an average diameter size of 10 μm or greater;
isolating the identified region of the vessel by occluding an upstream region and a downstream region from the identified region; and
delivering an agent within the isolated identified region under pressure to drive the agent into the vasa vasorum, wherein the pressure is maintained at 150 mmHg or less.

26.-38. (canceled)
